(19) United States
(12) Patent Application Publication
(10) Pub. No.: US 2025/0120749 A1
(43) Pub. Date: Apr. 17, 2025

(54) ADD-ON COUPLER FOR MEDICAL IMPLANT REVISION

(71) Applicant: NEXUS SPINE, LLC, Salt Lake City, UT (US)

(72) Inventors: Peter A. Halverson, Salt Lake City, UT (US); David T. Hawkes, Salt Lake City, UT (US)

(21) Appl. No.: 18/917,893

(22) Filed: Oct. 16, 2024

Related U.S. Application Data
(60) Provisional application No. 63/544,585, filed on Oct. 17, 2023.

(51) Int. Cl. A61B 17/70; A61B 17/00; A61B 17/56; A61F 2/30; A61F 2/44

(52) U.S. Cl. CPC A61B 17/7023; A61F 2/4455; A61B 2017/00477; A61B 2017/564; A61F 2002/3069; A61F 2002/4415

(57) ABSTRACT

Add-on couplers and coupler systems for use in surgical procedures and methods for utilizing add-on couplers in surgical procedures are provided. In some implementations, the add-on coupler includes a coupler receptacle configured to receive and couple to a first coupler, the first coupler being configured, in some cases, to couple to an implant anchor. In some implementations, the add on coupler includes a rod receptacle configured to receive a rod of a second coupler. In some cases, the second coupler and add-on coupler are coupled to an existing component from an earlier surgical procedure, thereby removing the need to remove the existing component prior to installing a new component. Additional implementations are described.

### RELATED APPLICATION

This application claims priority to U.S. Provisional Application Ser. No. 63/544,585 (Attorney Docket No. 23845.161), entitled ADD-ON COUPLER FOR MEDICAL IMPLANT REVISION, and filed Oct. 17, 2023, the contents of which are incorporated herein by reference in their entirety.

### FIELD

The present disclosure relates to surgical procedures, and more particularly to extension of a surgical construct to an adjacent area, in which extension of a medical implant is necessary or desirable.

### BACKGROUND

Treatment of certain conditions often requires surgical intervention. Some surgical procedures require the use of one or more implants. Many pathological conditions require multiple surgical interventions and some implant revisions. Revisions with some currently available implants can be difficult to perform, require removal and replacement of the original parts, and can otherwise be burdensome. This can lead to lengthy surgeries, unnecessary damage to the patient (for example, when screws are removed, they often must be replaced with screws of a larger bore, leading to more damage to the patient's bones), increased chances of the patient rejecting the new implant, longer recovery times, and other complications.

Thus, while techniques currently exist that are used to revise implants, challenges still exist, including those listed above. Accordingly, it would be an improvement in the art to augment or even replace current implants and techniques with other implants and techniques.

### SUMMARY

While medical implants, such as spinal implants, can provide much-needed treatment of medical issues, additional medical issues can manifest after the initial surgical intervention. Regarding spinal implants in particular, it is sometimes the case that a patient will require spinal fusion of several vertebrae, then subsequently (e.g., months to years later) require fusion of one or more additional vertebrae in proximity to the already fused vertebrae. When this is the case, revision surgery traditionally required the old implant to be removed and a new longer implant (spanning the additional vertebrae to be fused) to be installed. Removal of the old implant and installation of the new implant in some cases would cause extensive surgical exposure, stress, pain, and even damage to the patient's spine.

Moreover, some surgical procedures would be simplified if the implants allowed for a surgeon to treat specific conditions individually, rather than collectively. For example, reducing a spondylolisthesis at one level and securing those vertebrae in place before treating the adjacent level would, in some cases, be advantageous. Also, placing screws with different trajectories at different levels could help. Furthermore, changing the stiffness of the implants for each respective segment of the spine would, in some cases, be useful. Additionally, eliminating intra-operative rod bending can, in some cases, simplify the procedure.

In accordance with some implementations, the systems and methods disclosed herein allow for revision of an implant (e.g., to allow fusion of adjacent spinal vertebrae) without removing the existing implant. For example, some implementations provide a joining component configured to join an existing implant component (e.g., a first coupler) to a new implant component (e.g., a second coupler). In some implementations, the joining component includes an add-on coupler.

According to some implementations, the add-on coupler has one or more coupler receptacles configured to receive the first coupler. In some cases, the add-on coupler is configured to form a tight fit, such as a snap fit or interference fit, with the first coupler. Indeed, in some cases, the coupler receptacle has a size and shape (e.g., an interior geometry) approximately equivalent to a size and shape (e.g., an exterior geometry) of the first coupler. Thus, some implementations of the add-on coupler snap onto the exterior of the first coupler, thereby providing an extension to the first coupler without the need to remove the first coupler.

According to some implementations, the described add-on coupler has one or more rod receptacles configured to receive the second coupler (e.g., a rod of the second coupler). In some cases, the rod receptacle includes a neck into which the rod can be inserted. Some implementations allow the rod to be inserted at a variety of angles. According to some implementations, the neck has a slit formed therein that can be compressed (e.g., the size of at least part of the slit can be reduced) to thereby decrease the diameter of the neck, consequently locking the rod in place.

According to some implementations, a collar is slidably disposed on the neck, such that moving the collar toward an end of the neck causes the slit to compress. Accordingly, some iterations allow for the rod to be locked in place by sliding the collar from a base of the neck toward the end of the neck.

In accordance with some implementations, the described add-on coupler is configured to be used with rods that have different stiffnesses, different diameters, different shapes, or any other suitable characteristic, such that one or more characteristics of the rod change from one level to another level.

Some implementations include one or more tools for attaching the add-on coupler to one or more of the first coupler and the second coupler. For example, some implementations include one or more anchor lockers configured to couple the add-on coupler to the first coupler (e.g., by pressing the coupler receptacle onto the first coupler to snap it into place). Some implementations include a rod locker configured to couple the add-on coupler to the second coupler (e.g., by sliding the collar toward the end of the neck to lock the rod in place).

As the skilled artisan will recognize, many additional components, features, and variations on the foregoing systems and methods are possible. Accordingly, additional implementations are described below.

### DESCRIPTION OF THE DRAWINGS

The objects and features of the present disclosure will become more fully apparent from the following description and appended claims, taken in conjunction with the accompanying drawings. Understanding that these drawings depict only typical embodiments of the disclosed systems and methods and are, therefore, not to be considered limiting of its scope, the systems and methods will be described and explained with additional specificity and detail through the use of the accompanying drawings in which:

FIG. 1 shows a perspective view of a coupler system including an add-on coupler, in accordance with a representative embodiment;

FIG. 2A shows a side perspective view of the add-on coupler, in accordance with a representative embodiment;

FIG. 2B shows a front perspective view of the add-on coupler, in accordance with a representative embodiment;

FIG. 3A shows a side perspective view of the add-on coupler coupled to a coupler, in accordance with a representative embodiment;

FIG. 3B shows a front perspective view of the add-on coupler coupled to the coupler, in accordance with a representative embodiment;

FIGS. 4A-4E depict a process of assembling a coupler system by installing the add-on coupler, in accordance with some representative embodiments;

FIG. 5 shows a coupler system with an add-on coupler having an alternate configuration, in accordance with a representative embodiment; and

FIGS. 6A-6D show coupler systems with add-on couplers having additional alternative configurations, in accordance with representative embodiments.

### DETAILED DESCRIPTION

Add-on couplers and coupler systems for use in surgical procedures and methods for utilizing add-on couplers in surgical procedures are provided. In some implementations, the add-on coupler is configured to allow a new surgical implant component to be coupled to an existing component from an earlier surgical procedure, thereby removing the need to remove the existing component prior to installing a new component.

A description of some embodiments will now be given with reference to the FIGS. It is expected that the present systems and methods may take many other forms and shapes, hence the following disclosure is intended to be illustrative and not limiting, and the scope of the disclosure should be determined by reference to the appended claims.

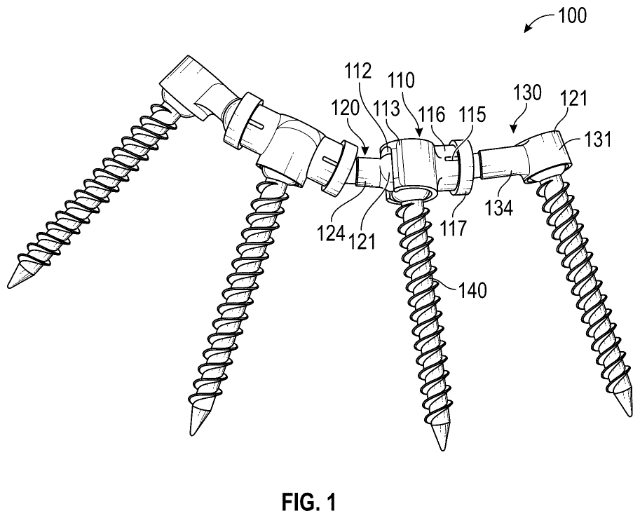

With reference generally to FIGS. 1-3B, some embodiments of the systems and methods disclosed herein include one or more coupler systems 100. Although the coupler system 100 can include any system used in connection with one or more medical implants (of any suitable kind) that are used in any portion of the body of a patient, some embodiments of the coupler system 100 relate to spinal implants. In this regard, after a patient has received a spinal surgery requiring the placement of one or more spinal implants, it is sometimes the case that the patient's spinal vertebrae adjacent to the implant site later require treatment, such as spinal fusion to join them to previously fused vertebrae. Typically, when this occurs, a provider removes the old implant and installs a new, larger implant that covers the additional treatment area. However, removal of an existing implant can cause problems, such as weakening of bone, damage to a patient's spine, pain, surgical complications, and other minor to severe problems.

Some embodiments of the coupler system 100 are configured to allow for one or more add-on couplers 110 to be joined to an existing implant, thereby removing the need for removing the old implant prior to installing a new one. Accordingly, some embodiments of the coupler system 100 include one or more add-on couplers 110.

Where the coupler system 100 includes one or more add-on couplers 110, the add-on coupler can include any suitable coupler configured to couple an existing implant to a new implant, or an existing portion of an implant to a new portion of an implant. In this regard, add-on couplers can be joined to an existing implant in any suitable manner, such as via one or more nails, screws, pins, bolts, staples, eyelets, magnets, hook-and-loop fasteners, adhesives, welds, interference fits, snap fits, friction fits, tongue-and-groove connections, snaps, ties, rivets, stakes, crimps, mechanical engagements, wire ties, threads or any other suitable coupling mechanism or mechanisms. By way of non-limiting illustration, FIG. 1 shows a coupler system 100 with an add-on coupler 110 configured to couple to an existing coupler 120 via a snap fit, a compression fit, a friction fit, or any other suitable type of fit that is capable of selectively locking the add-on coupler 110 in place with respect to the existing coupler 120.

In some embodiments, the add-on coupler 110 is configured to fixedly couple to the existing coupler 120 at only a single orientation (e.g., such that when the add-on coupler is coupled to the existing coupler, a longitudinal axis of the add-on coupler can only be disposed at a single angle with respect to a longitudinal axis of the existing coupler). In some embodiments, however, the add-on coupler is configured to couple to the existing coupler at multiple or many different orientations, such that the add-on coupler functions as a poly-axial coupler. In some such embodiments, the add-on coupler acts as a poly-axial coupling that is able to bridge from an end of the existing coupler to the best (or other desired) bone of the adjacent revision site. Thus, some such embodiments of the add-on coupler can be readily oriented and locked in place so as to be tailored to a particular patient and that patient's needs. In this regard, some embodiments of the described add-on coupler are extremely robust-thus allowing the patient to live a relatively normal life, without concern of damaging or changing a configuration of the coupler system. Moreover, while some embodiments of the described add-on coupler are poly-axial, some such embodiments are also low bulk (as shown in FIG. 1), such that the add-on coupler is relatively comfortable for the patient and so as to prevent the coupler from damaging surrounding muscle and tissue.

According to some embodiments, the add-on coupler 110 includes one or more receptacles. The receptacles can include any configuration that is suitable to allow for reception of another component, including one or more cavities, grooves, recesses, clips, divots, holes, passages, cups, sockets, voids, threads, or other receiving components. Although the add-on coupler can include any number of any suitable type of receptacles, some embodiments include one or more first receptacles 111 configured to receive a portion of a first implant (or a first portion of an implant) and one or more second receptacles 114 configured to receive a portion of a second implant as (or a second portion of an implant). As embodiments with a first receptacle and a second receptacle may be particularly useful for coupling one or more new implant components to one or more existing implant components, the first and second receptacles are each discussed in more detail below. For convenience, the first receptacle is referred to generally as a coupler receptacle, and the second receptacle is referred to generally as a rod receptacle, but it is important to note that any of the receptacles discussed herein can have any of the features of any of the other receptacles, and that the various receptacles are not limited to receiving couplers or rods respectively. Indeed, in some cases, the coupler receptacle and the rod receptacle are interchangeable, and in some cases their positions or roles are reversed.

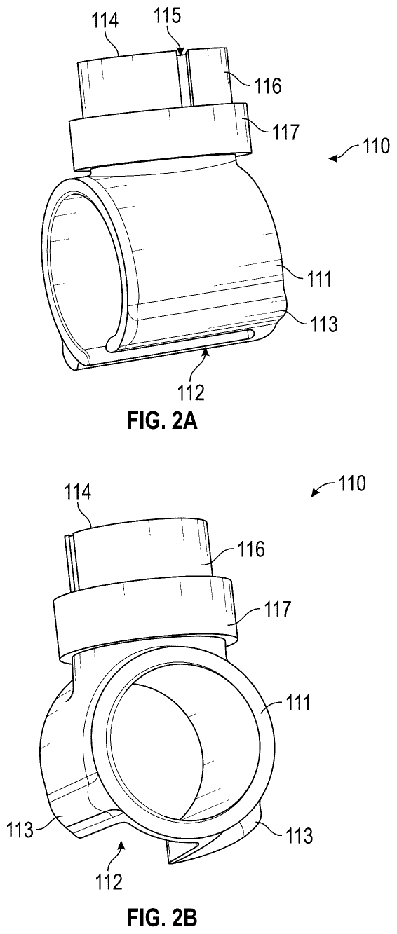

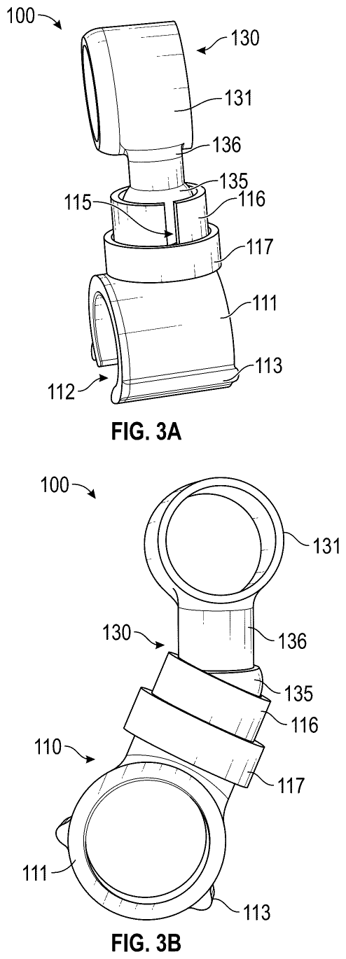

According to some embodiments, the add-on coupler 110 includes a coupler receptacle 111 that is configured to receive a portion of an existing implant. The coupler receptacle can include any component configured for receiving a portion of an implant, including one or more cavities, grooves, recesses, clips, divots, holes, passages, cups, sockets, voids, or other receiving components. The coupler receptacle can also be configured to receive any portion of an existing implant, such as one or more couplers, anchors, rods, necks, collars, processes, bodies, faces, surfaces, screws, tulips, balls, enlarged portions, catches, or any other portions of an implant. In some embodiments, the coupler receptacle is configured to receive a coupler 120 (or a portion of a coupler). By way of non-limiting illustration, FIGS. 2A-2B show an add-on coupler 110 having a coupler receptacle 111 configured to receive an existing coupler (e.g., by being configured to fit over an anchor receptacle 121 of a coupler 120 as shown in FIG. 1).

Where the add-on coupler 110 comprises one or more coupler receptacles 111, the coupler receptacle 111 can have any shape suitable for receiving one or more portions of one or more existing implants. For example, some embodiments of the coupler receptacle 111 include a hollow body, having a cavity in which a part of an implant (e.g., an anchor receptacle 121) is configured to fit. In some cases, the hollow body is substantially cylindrical (e.g., having a substantially circular cross section). In some cases, the hollow body has a cross section of another shape, such as being semi-circular, triangular, square, rectangular, trapezoidal, pentagonal, hexagonal, star-shaped, T-shaped, polygonal, or any other regular or irregular shape. By way of non-limiting illustration, FIGS. 2A-3B show various add-on couplers 110 having coupler receptacles 111 that are substantially cylindrical (with a substantially cylindrical interior geometry and a substantially cylindrical exterior geometry, except that the exterior of some embodiments further include one or more processes 113, as discussed in more detail below). Additionally, in some embodiments, the add-on coupler comprises one or more rounded edges, has one or more smooth surfaces, is relatively small in size and is otherwise configured to be ergonomic such that it fits with little to no trespass of any facet joints of the patient or irritation to the soft tissues.

In some embodiments, the coupler receptacle 111 is configured to selectively snap in place over an existing component (e.g., an anchor receptacle 121 of an existing coupler 120), thereby forming a strong connection requiring many pounds of force (e.g., between 10 and 10,000 pounds of force (e.g., 0 newtons and 44,448.2 newtons), or any subrange thereof) in order to separate the coupler receptacle 111 from its complementary component. For example, some embodiments are configured to form a coupling that requires at least 100 pounds of force (444.8 newtons), at least 200 pounds of force (889.6 newtons), at least 300 pounds of force (1,334.5 newtons), at least 400 pounds of force (1,778.3 newtons), at least 500 pounds of force (2,224.1 newtons), at least 750 pounds of force (3,336.2 newtons), at least 1000 pounds of force (4,448.2 newtons), at least 1500 pounds of force (6,672.3 newtons), at least 2000 pounds of force (8,896.4 newtons), or at least 2500 pounds of force (11,120.6 newtons) to separate the add-on coupler from the existing coupler. In this regard, it should be noted that the force required for a bone-to-hardware interface to fail is, in some cases, about 400 newtons. Thus, in some embodiments, the force required to move the add-on coupler with respect to the existing coupler (once the add-on coupler is selectively locked in place) exceeds (and in some embodiments, significantly exceeds) the strength of the bone interface. Also, it should be noted that implants can be loaded repeatedly for the life of the patient and can, as a result, fatigue. That said, some embodiments of the described add-on coupler can withstand forces (e.g., between 10 and 10,000 pounds of force (e.g., between 0 newtons and 44,448.2 newtons), or any subrange thereof) for millions of cycles. Indeed, in some embodiments, the described add-on coupler is configured to exceed an 89.9 pound (400 newton) load for millions of cycles. Thus, while many existing implants loosen with time, some embodiments of the descried add-on coupler are configured to significantly reduce or even eliminate implant loosening and construct instability.

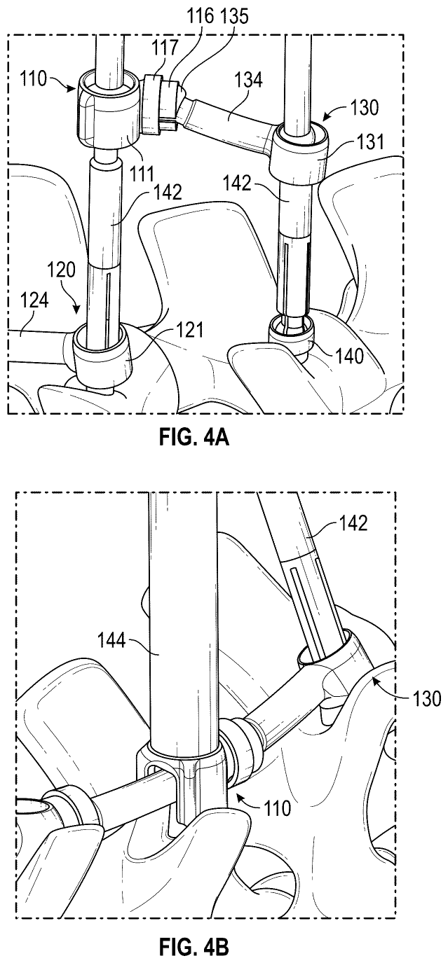

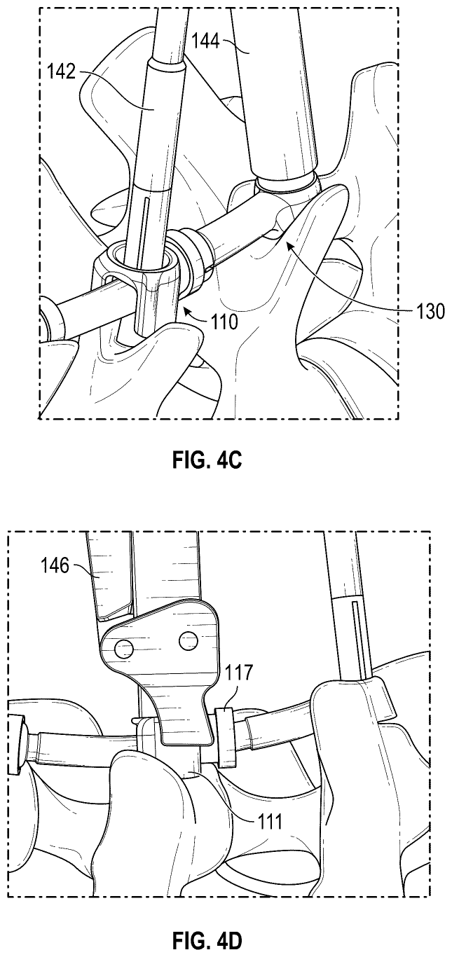

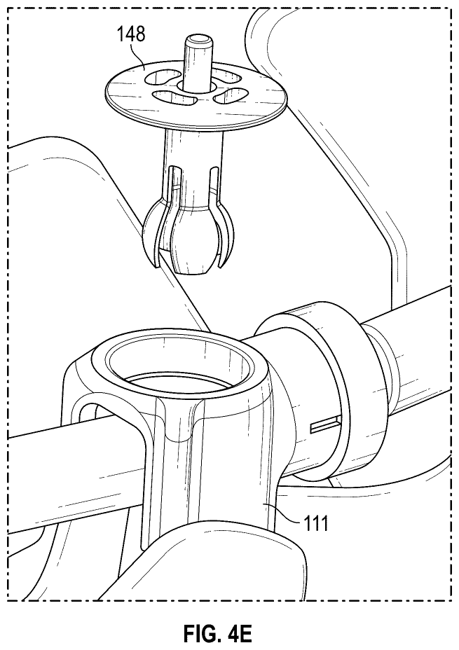

In some embodiments, the coupler receptacle 111 includes one or more catches, such as one or more protrusions, rings, grooves, lips, recesses, barbs, ribs, teeth, knurled surfaces, roughened surfaces, threads, or any other suitable feature for effectuating a snap fit, frictional fit, or other interference fit. In some embodiments, the complementary component to which the coupler receptacle is configured to couple (e.g., a coupler 120) includes one or more complementary catches, such as one or more grooves, slots, indentations, ring, protrusions, recesses, teeth, threads, or any other suitable feature for engaging with the one or more catches of the coupler receptacle. That said, in some embodiments, the coupler receptacle 111 does not include a protrusion and is not otherwise configured snap fit with a complementary component, but rather the add-on coupler 110 is configured to couple to a complementary component (e.g., a coupler 120) via a connector, such as a rivet (e.g., the rivet 148 shown in FIG. 4E or another type of rivet) or any other suitable pin, screw, snap, clamp, or other type of connector. In some embodiments, the coupler receptacle 111 is configured to form a snap fit, frictional fit, or other interference fit with a complementary component, and it is additionally configured to be further secured in place via a connector.

In some embodiments, the coupler receptacle 111 includes one or more openings 112. The opening can include any suitable opening, groove, slot, passage, hole, slit, socket, mouth, or other subtraction formed in a wall or any other suitable portion of the coupler receptacle to thereby allow one or more other components to extend therethrough. In some cases, the opening aligns with a feature of the existing implant to thereby accommodate such portion. For example, in some cases, the opening allows for a portion of an existing coupler 120 (e.g., a rod 124) to extend laterally away from the coupler. In some embodiments, the opening allows the body of the coupler receptacle to be somewhat compliant. For example, in some cases the opening allows the coupler receptacle to snap in place over another component (such as an anchor receptacle 121), where all or part of the other component has a diameter equal to or greater than all or part of a diameter of the cavity of the coupler receptacle 111.

The opening 112 can have any suitable shape allowing it to accommodate one or more implant components or to impart one or more desired properties to the add-on coupler 110 (such as a measure of flexibility). For example, the opening can be generally circular, semi-circular, triangular, square, rectangular, trapezoidal, pentagonal, hexagonal, star-shaped, T-shaped, U-shaped, V-shaped, polygonal, or any other regular or irregular shape. In some embodiments, the opening is substantially U-shaped (e.g., V-shaped, U-shaped, generally the shape of three sides of a rectangle, semi-circular, or another similar shape). In some embodiments, the opening extends only partway along the body of the coupler receptacle 111. In other embodiments, the opening extends through an entire wall of the receptacle (e.g., becoming a slot or slit). While the opening may have any suitable orientation, some embodiments of the opening are parallel or substantially parallel (e.g., within 10 degrees of parallel) with a longitudinal axis of the coupler receptacle. Further, the opening can have any suitable size. In some cases, the opening is between 1 mm and 3 cm (or any subrange thereof, such as 1 cm+1 cm). In some cases, the opening spans between 1 degree and 180 degrees of the body of the coupler (or any subrange thereof, such as 90 degrees±35 degrees, 75 degrees±25 degrees, or any other suitable subrange). Some embodiments of the opening have a width (e.g., a distance from one leg or edge of the “U” shape to the other) that is approximately equal to (or just barely larger than) a diameter of a rod 124 of a coupler 120. In some embodiments, the opening has a height (e.g., a distance from the bottom of the “U” shape to the open top) of between 50% and 99% of a height of the add-on coupler or a height of the coupler receptacle (or any subrange thereof, such as 90%+5%, 85%+12%, or any other suitable subrange).

By way of non-limiting illustration, FIG. 1 shows an add-on coupler 110 having an opening 112 of sufficient size to allow a rod 124 of an existing implant coupler 120 to extend therethrough, the opening being substantially U-shaped, facing a bottom of the add-on coupler and being continuous with a bottom opening through which an anchor 140 extends.

In some embodiments, the opening 112 is sized with tight tolerances such that it tightly fits the existing coupler (a rod 124) so that the add-on coupler can only be placed on the existing coupler (e.g., anchor receptacle 121) at one angle of rotation with respect to the existing coupler. In some embodiments, however, the opening is wider than the corresponding portion of the existing coupler, such that the add-on component can be placed on the existing coupler at one or many different rotational configurations with respect to the existing coupler. Again, however, once the add-on coupler is selectively snapped (or locked) in place, the rotational configuration of the add-on coupler with respect to the existing coupler is also locked in place.

In some embodiments, the coupler receptacle 111 includes one or more processes 113. While the processes can include any protrusion or other component configured to increase a rigidity of all or part of the coupler receptacle or to perform any other suitable function, in some embodiments, the processes include one or more lips or ridges formed along a portion of the coupler receptacle. The processes can have any suitable shape, including being linear, wavy, curved, polygonal, rounded, or otherwise having any suitable shape. In some embodiments, the processes are elongated, and in some cases, a longitudinal axis of one or more of the processes runs substantially parallel to a longitudinal axis of the coupler receptacle (e.g., running between a top portion and a bottom portion of the coupler receptacle). The processes can also be disposed in any suitable location in relation to the add-on coupler 110. For example, in some embodiments, the processes are formed along all or part of a perimeter of the opening 112. In some cases, the processes form a thicker portion of the body of the coupler receptacle, thereby decreasing the compliance thereof (e.g., forming a stronger coupling with the anchor receptacle 121 or with another complementary coupling component). Indeed, some embodiments of the processes are configured to increase a coupling strength of the add-on coupler with a complementary coupling component (e.g., an existing coupler 120 of an existing implant) by 10 pounds to 5,000 pounds (or 44.5 newtons to 22,241.1 newtons), or any subrange thereof (e.g., 1,000 pounds±500 pounds (4,448.2 newtons±2,224.1 newtons), or any other suitable subrange) so as to increase the pounds of force needed to move the add-on coupler with respect to the existing coupler. By way of non-limiting illustration, FIGS. 1-3B show processes 113 bordering openings 112 of various embodiments of add-on couplers 110, with the processes extending along a height of the bodies of the add-on couplers 110.

In some embodiments, the add-on coupler 110 includes one or more rod receptacles 114. The rod receptacle can include any suitable component configured to receive any suitable portion of a new implant, or a new portion of an implant, desired to be coupled to an existing implant. For example, the rod receptacle of some embodiments is configured to receive one or more couplers, processes, rods, flanges, necks, collars, clips, rivets, bodies, surfaces, balls, catches, couplers, or other implant components. In some embodiments, the rod receptacle is configured to receive a rod (e.g., a rod 134 of a new coupler 130 configured to secure a new anchor to the add-on coupler 110, thereby attaching the new anchor to the old implant, forming a unitary coupler system 100 that includes one or more new implants and one or more old implants, without the need for removing the old implants). As with other receptacles discussed herein, the rod receptacle can include any suitable type of receptacle, including one or more cavities, grooves, recesses, clips, divots, holes, passages, cups, sockets, catches, couplers, or other receiving components. By way of non-limiting illustration, FIGS. 2A and 2B show an embodiment of an add-on coupler 110 with a rod receptacle configured to receive a rod of a new implant (e.g., the rod 134 of the new coupler 130 as shown in FIG. 1).

In some embodiments, the rod receptacle 114 includes a neck 116. The neck can include any suitable component configured to provide a portion of the receptacle in which to house a rod or another component. In some cases, the neck 116 is substantially cylindrical (although it can be any other suitable shape, such as by having a cross section that is circular, semi-circular, spherical, semi-spherical, triangular, square, rectangular, trapezoidal, pentagonal, hexagonal, star-shaped, T-shaped, polygonal, prismatic, or any other regular or irregular shape). In some cases, the neck 116 forms a cavity into which a component (e.g., a rod 134 of a coupler 130 of a new implant) can be inserted.

In some embodiments, the neck 116 extends a distance from the body of the add-on coupler 110. The distance can be any suitable distance, such as between 1 mm and 10 cm (or any subrange thereof, such as 1 cm+0.8 cm). In some cases, the neck extends a distance between 50% and 200% of a diameter of the coupler receptacle 111 (or any subrange thereof, such as 100%+25%). Similarly, the neck can have any suitable diameter, such as between 1 mm and 2 cm, or any subrange thereof (e.g., 0.8 cm+0.5 cm). In some embodiments, the diameter of the neck is between 50% and 200% of a width of the opening 112 (or any subrange thereof, such as 100%±50%, 100%±25%, or any other suitable diameter). Thus, in some embodiments, the opening and the rod receptacle are configured to accommodate rods or other components of identical or similar sizes, while in other embodiments, the opening and the rod receptacle are configured to accommodate components of different sizes. This allows for flexibility in a revision surgery to accommodate existing components and select ideal new components. By way of non-limiting illustration, FIGS. 1-3B show add-on couplers 110 having substantially cylindrical necks 116 extending away from the bodies of such add-on couplers 110.

In some embodiments, the neck 116 includes one or more thinned, perforated, bent, slit, or other portions that are configured to allow the opening of the rod receptacle 114 to be crimped, tightened, reduced in size, expanded, flared, or otherwise modified in size to accommodate coupling with desired components (e.g., a rod 134 of a coupler 130 of a new implant) into the neck. Indeed, in some embodiments, the neck includes one or more slits 115. Similar to the opening 112 of the coupler receptacle 111, the slit 115 can include any feature that gives the neck more flexibility or allows it to accommodate one or more components. For example, in some embodiments, the slit allows the neck to expand to fit a component (e.g., a rod 134) within the neck's cavity. In another example, some embodiments of the slit include one or more notches, grooves, passages, indentations, scores, recesses, or any other suitable flexibility or deformable features. While the slit can be positioned in any suitable location on the neck, some embodiments of the slit are positioned toward an end of the neck opposite the body of the add-on coupler 110 (and in some cases, the slit is formed right to the end, thereby being continuous with an opening of the receptacle at the end of the neck). The slit can also be oriented in any suitable manner (e.g., at any angle with respect to any other component), but in some cases it is substantially aligned with a longitudinal axis of the neck (e.g., it is substantially parallel with the neck). Additionally, while the slit can have any suitable width (e.g., extending up to 180 degrees around the neck), some embodiments of the slit (or one or more slits) are discrete (e.g., between 0.1 degree and 15 degrees, or any subrange thereof, such as 3±2.5 degrees). Similarly, the slit can have any suitable length, such as between 1% and 100% of the length of the neck, or any subrange thereof (e.g., 50%±20%, 50%+10%, or any other suitable subrange). By way of non-limiting illustration, FIGS. 1, 2A, and 3A show some embodiments of add-on couplers 110 with necks 116 having slits 115 formed therein, the slits being fairly discrete and extending from approximately a middle portion of such necks to an end portion of such necks (with a portion such necks being obscured in the FIGS. by respective collars 117, as discussed more below).

The rod receptacle 114 can be selectively locked to fixedly couple the rod receptacle with one or more desired components (e.g., a rod 134 of a coupler 130 of a new implant) in any suitable manner, including through the use of one or more collars, welds, adhesives, pins, pawls, frictional engagements, mechanical engagements, catches, or in any other suitable manner. In some embodiments, however, the rod receptacle 114 includes one or more collars 117. The collar can include any object or feature useful for locking a component (e.g., a rod 134) in place once the component is inserted into neck 116. For example, in some cases, the collar 117 includes one or more rings, sleeves, washers, collets, crimps, clips, clamps, clasps, adjustable bands, worm gear clamps, or other securement devices.

In some embodiments, the collar 117 (e.g., a ring) is configured to slide along the neck 116. In some embodiments, a diameter of the collar is slightly less than a diameter of at least a portion of the neck (e.g., proximate the slit 115). In some embodiments, the diameter of the collar is adjustable. In some cases, after a component (e.g., a rod 134) is inserted into the neck, the collar can slide toward an open end of the neck, thereby pressing the end portions of the neck together around the component (e.g., around a flange 135 of the rod 134), thereby forming a strong frictional or interference fit. In some cases, sliding the collar toward the coupling end of the neck 116 causes the neck body near the slit 115 to press together (e.g., collapsing or shrinking the slit 115), such that the neck 116 presses in tighter around the inserted component. By way of non-limiting illustration, FIG. 1 shows, in accordance with some embodiments, an add-on coupler 110 with a collar 117 slidably disposed on a neck 116, the collar being disposed near the end of the neck 116, thereby causing a slit 115 to compress, consequently locking a rod 134 in place.

Where the add-on coupler 110 comprises one or more collars 117, the collar can be configured to be selectively locked in place and to remain in place (e.g., to keep a rod 134 or other component locked in the neck 116) in any suitable manner. Indeed, in some embodiments, the collar is held in place towards an end of the neck to lock a component (e.g. a rod) in place via friction; pressure; an adhesive; one or more protuberances, projections, catches, barbs, recesses, or any other suitable features on the neck or collar; fasteners; or in any other suitable manner. Indeed, in some embodiments, the collar is held in place to lock a component in the neck though friction. Moreover, in some cases, in order to lock the collar in place, the collar is pushed past a bulging portion of the neck (e.g., a portion of the neck that comprises a relatively wide portion of the component (e.g., the rod) that is being locked in the neck) such that it would take a significant amount of force to push the collar back away from the end of the neck and over the bulge to release the component.

The add-on coupler 110, as discussed herein, can be implemented in any suitable system in which one or more implants (or a portion of an implant) is connected to another implant (or to another portion of the implant). That said, some embodiments of the add-on coupler are particularly suited for use in a coupler system 100 in which the add-on coupler 110 is used to couple a first coupler 120 (e.g., a coupler of an old or existing implant) to a second coupler 130 (e.g., a new coupler or revision coupler as may be used in revision surgery).

As an illustrative example of a suitable system, some embodiments of the system 100 include a first coupler 120. In some cases, the first coupler includes (e.g., is, or is part of, or contains) an existing implant. In some cases, the existing implant was installed as part of a prior spinal fusion surgery targeting a number of spinal vertebrae. Some embodiments of the system include a second coupler 130. In some cases, the second coupler includes (e.g., is, or is part of, or contains) a new implant. In some cases, the new implant is installed as part of a revision surgery in which additional spinal vertebrae are to be fused (e.g., in addition to the vertebrae fused as part of the prior surgery). In some embodiments, the add-on coupler joins the second coupler to the first coupler without the need to remove the first coupler from the patient, which can thereby reduce damage, pain, recovery time, and other complications that can be associated with removing the first coupler. By way of non-limiting illustration, FIG. 1 shows a coupler system 100 having a first coupler 120 joined to a second coupler 130 by an add-on coupler 110.

According to some embodiments, the first coupler 120 includes one or more first anchor receptacles 121. Further, some embodiments of the second coupler 130 include one or more second anchor receptacles 131. The anchor receptacles can include any components configured to receive one or more implant anchors 140. For example, some embodiments of the anchor receptacles have a body with a passage for receiving an anchor formed therethrough. In some cases, the body shares one or more characteristics of the add-on coupler 110. For example, in some cases the body has a generally cylindrical housing with a hollow bore, although it can have any suitable shape (e.g., having a cross section that is circular, semi-circular, triangular, square, rectangular, trapezoidal, pentagonal, hexagonal, star-shaped, T-shaped, polygonal, or any other regular or irregular shape). As another example, an internal surface of the anchor receptacles includes one or more catches, such as one or more protrusions, rings, grooves, lips, recesses, barbs, ribs, teeth, knurled surfaces, roughened surfaces, or any other suitable feature for effectuating a snap fit, frictional fit, or other interference fit (e.g., with an implant anchor).

The anchor receptacles 121, 131 can also have any suitable size. For example, the anchor receptacles can have a diameter that is approximately equivalent to a diameter of an internal bore of a coupler receptacle 111 of an add-on coupler 110. In some embodiments, the anchor receptacles have a diameter that is slightly less than the diameter of the internal bore of the coupler receptacle, but in some embodiments, the diameter of the anchor receptacles is slightly greater than the diameter of the internal bore of the coupler receptacle, thereby requiring the coupler receptacle to expand (e.g., as enabled by the opening 112) to accommodate the coupler. In some cases, this forms a strong connection requiring a strong force (e.g., a force that is greater than is ordinarily exerted upon the spine under ordinary physiological conditions) to separate. Indeed, in some embodiments, this described connection is stronger than the connection between the implant anchors 140 and the corresponding bone.

The anchor receptacles 121, 131 can be configured to receive any type of anchor 140. In particular, the anchor can include any suitable component for embedding in or securing another component to any portion of a patient. In some embodiments, the anchors include one or more nails, screws, pedicle screws, bolts, tulips, staples, eyelets, fasteners, stakes, wires, pins, rods, rivets, hooks, barbs, adhesives, implants, or any other suitable types of anchors. In some cases, the first anchor is configured to be anchored in a first spinal vertebra, and the second anchor is configured to be anchored in a second spinal vertebra. In some embodiments, the coupler system 100 or part thereof (alone or together with one or more additional components) joins the first anchor to the second anchor. By way of non-limiting illustration, FIG. 1 shows a first coupler 120 and a second coupler 130, each of which has an anchor receptacle 121, 131 in the form of a hollow cylinder with an anchor 140 in the form of a medical implant screw extending therethrough.

As briefly mentioned above, some embodiments of the system 100 include one or more rods 134. For example, some embodiments of the first coupler 120 include one or more first rods 124, and some embodiments of the second coupler 130 include one or more second rods 134. The rods can include any components configured to link together other components of the system. For example, some embodiments of the rods include one or more links, pipes, shafts, bars, stems, beams, dowels, or other linkage components. The rods can also have any suitable length, such as between 10 mm and 30 cm, or any subrange thereof (e.g., 3 cm+2 cm).

The rod 134 can be coupled to the couplers 120, 130 (e.g., the cylindrical bodies of the couplers) in any suitable manner. In some embodiments, the rods are coupled to the couplers via welding, adhesive attachment, friction or interference fit, mechanical engagement, or by any other suitable attachment mechanism. In some embodiments, at least one end of the rod 124, 134 is rigidly fixed to a coupler (e.g., welded, permanently attached, integrally formed, or otherwise rigidly fixed). In some embodiments, at least one end of the rod is selectively attachable to a coupler (e.g., through a selectively removable attachment mechanism, such as a tie, a clip, a clasp, a receptacle, a friction or interference fit, or another selective attachment mechanism. For example, in some embodiments, the rod is coupled to a coupler using a rod receptacle 114 and collar 117, as discussed above. By way of non-limiting illustration, FIG. 1 shows a first coupler 120 having a rod 124 with a first end that is fixedly attached to a body of the first coupler 120, the body of the first coupler 120 being disposed within a coupler receptacle of an add-on coupler 110, the rod having a second end that is selectively attached to an additional coupler via a coupler receptacle with a neck and a collar. FIG. 1 further shows a second coupler 130 having a rod 134 with a first end that is fixedly attached to a body of the coupler (i.e., the anchor receptacle 131) and a second end that is selectively secured to the add-on coupler 110 by being inserted into a neck 116 of a rod receptacle and secured therein via a collar 117.

In some embodiments, the rod 124, 134 is configured to be selectively secured to a coupler (e.g., the add-on coupler 110, the first coupler 120, or the second coupler 130) at a variety of different angles. For example, in some embodiments, the rod is configured to be disposed within a rod receptacle 114 at an angle of a practitioner's choosing, with the angle being selectively adjustable. To illustrate, in some embodiments, the rod is configured such that a user can position it substantially parallel to the neck 116 (e.g., extending orthogonally from an opening of the neck), or such that the user can position it at an angle with respect to the neck (e.g., between 0 degrees and 75 degrees, or any subrange thereof, such as between 0 and 45 degrees, 30 degrees+30 degrees, or any other suitable subrange). In some embodiments, the angle is selectively adjustable along three degrees of freedom (e.g., it can be an upward angle, a downward angle, a leftward angle, a rightward angle, or a combination of two of the foregoing). In some embodiments, the rod is configured to be locked in place (thereby being rigidly fixed at the selected angle) after the angle is selected. In some embodiments, the locking in place is strong enough that it would require many pounds of force (e.g., between 50 pounds of force (222.4 newtons) and 10,000 pounds of force (44,483.2 newtons), or any subrange thereof (e.g., 250 pounds of force (1,112.1 newtons)+150 pounds of force (667.2 newtons)) to change the angle or to unseat the rod.

Although the rod 124, 134 can be configured to be disposed at any angle with respect to the rod receptacle 114 in any suitable manner (e.g., by having a ball-and-socket joint or any variant thereof), some embodiments include one or more flanges 125, 135. In some embodiments, the rod receptacle (e.g., in the neck 116 of the add-on coupler 110 or another coupler component) is configured to receive the one or more flanges. The flange can include any polyaxial connecting component. Accordingly, the flange can have any geometry that allows it to behave generally as a ball of a ball-and-socket joint and to be locked in place. For example, it can be spherical, hemispherical, conical, paraboloid, toral, cylindrical, elliptical, or otherwise configured to form a tight fit within the rod receptacle such that it can be locked in place (e.g., by compression of the slit 115 in the neck via tightening or placement of the collar 117). By way of non-limiting illustration, FIG. 3A shows a flange 135 having a general paraboloid configuration, and FIG. 3B shows a flange 135 having a general toral or ring configuration, in each case allowing the rod 136 to be disposed at a variety of angles (in three degrees of motion) with respect to the neck 116, after which the collar 117 can be slid to the end of the neck, thereby tightly locking the construct in the selected configuration.

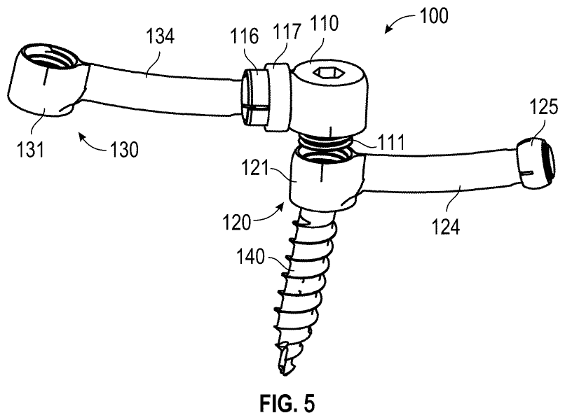

While some embodiments of the flange 125, 135 are smooth, some embodiments optionally comprise one or more protrusions, rings, grooves, lips, recesses, barbs, ribs, teeth, knurled surfaces, roughened surfaces, or any other suitable feature to help the flange be selectively locked in position with respect to the rod receptacle 114. By way of non-limiting illustration, FIG. 5 shows a flange 125 in substantially the form of a collet having a plurality of grooves formed therein.

With reference generally to FIGS. 4A-4E, some embodiments include a method for providing an add-on coupler 110, or for coupling a new implant to an existing implant (e.g., by attaching a new coupler 130 to an old coupler 120).

In some embodiments, the method includes forming, manufacturing, modifying, using, or otherwise obtaining or utilizing one or more components or use in connection with the add-on coupler 110. For example, in some embodiments, the method includes obtaining one or more add-on couplers 110 (or parts thereof, such as coupler receptacles 111, openings 112, processes 113, rod receptacles 114, slits 115, necks 116, or collars 117), old couplers 120 (or any parts thereof), new couplers 130 (or any parts thereof), anchors 140, wands 142, anchor lockers 144, rod lockers 146, rivets 148, or any other suitable components.

In some embodiments, the method includes coupling the new coupler 130 to the add-on coupler 110. In some embodiments, such coupling includes inserting a rod 134 of the coupler 130 (or a part thereof, such as a flange 135) into a rod receptacle 114 of the add-on coupler 110. In some embodiments, the coupling further includes sliding a collar 117 of the add-on coupler 110 along a neck 116 of the add-on coupler 110 in order to lock the neck around the rod 134 or flange 135. In some embodiments, this includes using a rod locker 146. The rod locker can include any component configured to assist or allow the coupling of the rod to the add-on coupler. For example, some embodiments include one or more handles and one or more heads. The handles can include any component configured to allow for holding, moving, or manipulating the rod locker. For example, in some embodiments, the handles are configured to be actuated (e.g., turned, squeezed, slid, or otherwise manipulated to cause the head to perform one or more functions). The head can be configured to perform any function useful for coupling of the rod to the add-on coupler, such as coupling to, holding, or moving one or more components (e.g., sliding the collar 117 in response to actuation). By way of non-limiting illustration, FIG. 4D shows a rod locker 146 configured to couple to a coupler receptable 111 (e.g., to an orifice of the coupler) and configured to force the collar 117 into a locking position.

In some embodiments, the method includes installing one or more new anchors 140 (e.g., by screwing them, driving them, or otherwise inserting them into a bone, an organ, a muscle, a tissue, or another suitable portion of a patient). That said, common practice often involves installing anchors into one or more bones (e.g., spinal vertebra) of a patient.

In some embodiments, the method includes coupling the new coupler 130 to the new anchor 140 (in any suitable manner, such as by bonding, welding, snapping, locking, sealing, using an adhesive, locking with a locking component such as a rivet 148, or otherwise coupling the new anchor 140 to the new coupler 130. Indeed, in some embodiments, as shown by FIG. 4E, the method includes inserting a rivet 148 into a coupler receptacle 111 to lock an anchor (not shown) to the coupler receptacle.

In some embodiments, the method includes coupling the add-on coupler 110 to an old coupler 120. For example, in some cases the method includes sliding a coupler receptacle 111 of the add-on coupler 110 over an anchor receptacle 121 of an old coupler 120. In some embodiments, this includes locking the add-on coupler 110 to the old coupler 120 (in any suitable manner, such as by bonding, welding, snapping, locking, sealing, using an adhesive, locking with a locking component such as a rivet, or otherwise coupling the add-on coupler 110 to the old coupler 120. Indeed, in accordance with some embodiments, as shown by FIG. 4C, the method includes sliding an add-on coupler 110 into place over an existing coupler (not shown) using a wand 142 (as discussed in more detail below) and snapping an add-on coupler into place over an existing coupler using an anchor locker 144.

In some embodiments, the method includes coupling one or more wands 142 to one or more existing or new components. The wand can include any component useful for installing any of the components discussed herein. For example, in some cases, one wand is coupled to (e.g., inserted into or otherwise selectively interfaces with) a newly installed anchor 140, and one wand is coupled to an existing anchor or an existing coupler 120. In some embodiments, the method includes sliding one or more components along the wands (e.g., in some cases, so that all or part of such components surrounds a wand and can travel along the wand like a ring around a stick). In some embodiments, the coupler receptacle 111 of the add-on coupler 110 slides down a wand coupled to an existing implant component, and the anchor receptacle 131 of the new coupler 130 slides down a wand coupled to a new implant component. In some cases, the add-on coupler is already coupled to the new coupler 130 when they are slid along the wands (although in some cases, they are coupled to each other after sliding along the wands).

In some embodiments, the method includes locking the add-on coupler 110 to the existing implant component. In some cases, this includes using an anchor locker 144. In some embodiments, the method includes locking the new coupler 130 to the new implant component using the anchor locker. In some embodiments, locking one or more components together using the anchor locker includes sliding the anchor locker over a wand 142 and pressing the components into place. In some embodiments, pressing the components into place includes engaging a snap or frictional fit. In some embodiments, the wand is removed prior to using the anchor locker.

In some embodiments, the anchor locker 144 includes one or more actuators, drivers, clamps, or otherer mechanisms by which the anchor locker may press the coupler receptacle 111 of the add-on coupler 110 and the anchor receptacle 121 of the coupler 120 together to engage them in a snap fit, a frictional fit, or another interference fit. In some embodiments, the anchor locker includes one or more tools for coupling components of a surgical construct, such as one or more tools disclosed in U.S. Pat. No. 9,801,667 B2 (titled “Instruments, Tools, and Methods for Presson Pedicle Screws”), which is incorporated by reference herein in its entirety.

In some embodiments, the method of locking the add-on coupler 110 to the first coupler 120 (e.g., using an anchor locker 144) includes applying an instrument with a functional area (e.g., an instrument head, or another portion of the instrument configured to contact or otherwise lock the add-on coupler into place) with a diameter that does not exceed the size of the diameter of the add-on coupler. Accordingly, in some embodiments, the instrument (or the functional area thereof) does not extend outwardly outside of the footprint of the add-on coupler. Thus, in some embodiments, the add-on coupler can be coupled to the first coupler without the instrument contributing to the radial bulk of the implant (accordingly, an easier, safer, and more comfortable operating environment can be provided).

In some embodiments, the method includes locking the components in place by inserting or engaging a connector, such as a rivet, pin, pawl, screw, or any other suitable type of connector. Indeed, in some embodiments, the method includes locking the components in place by inserting or engaging a rivet (e.g., the rivet 148 shown in FIG. 4E or another rivet). In some embodiments, the method includes locking the components in place with a rivet system like the one described in U.S. patent application Ser. No. 18/236,730 (titled “Systems and methods for securely attaching an anchor to an implant body”), which is incorporated by reference herein in its entirety. In some embodiments, the method includes using a single rivet to couple together the anchor 140, the anchor receptacle 121 of the coupler 120, and the coupler receptacle 111 of the add-on coupler 110. In this regard, it should be noted that by allowing the add-on coupler to be initially placed in the patient such that it is movable (or in a floppy state) before being locked in place with respect to one or more other components of the system 100, the add-on coupler can be tailored to, and allow the system 100 (or a portion thereof) to take on the shape of the patient's spine. Accordingly, in some cases, the add-on coupler is configured to be locked in place (or made rigid) to hold the spine in its proper and natural anatomical position without causing residual stress. Thus, some embodiments of the described systems and methods are configured to reduce or even eliminate pain from unintended residual stress. In some embodiments, however, the add-on coupler and one or more other components are configured to be locked together or in place (e.g., made rigid) prior to attachment to the spine to apply manipulating forces to the spine. Thus, some embodiments of the described systems and methods allow surgeons to have multiple options when installing the add-on coupler, to help the surgeon obtain the best results.

Any of the components discussed herein (e.g., the add-on coupler 110; the coupler receptacle 111; the process 113; the rod receptacle 114; the neck 116, 126, 136; the collar 117; the anchor receptacle 121, 131; the rod 124, 134; the flange 125, 135; the first coupler 120; the second coupler 130; the anchors 140; the wand 142; the anchor locker 144; the rod locker 146; the rivet 148; or any other component) can be formed from any suitable material. For example, in some embodiments, one or more components includes one or more types of metal (e.g., iron, copper, tin, steel, aluminum, titanium, zirconium, brass, cobalt chrome, or any other biocompatible metal or metal alloy), glass, plastic, ceramic, carbon fiber, polymer material, wood, natural material, synthetic material, or any other suitable material. Indeed, in some embodiments, the described components comprise titanium. Generally speaking, the materials used are biocompatible (e.g., non-toxic). In some cases, the materials optionally include one or more antibacterial, antiviral, or antifungal coatings or properties to reduce the risk of infection. In some cases, a lubricant is used to more easily engage a coupling (e.g., a snap fit), which can include any suitable biocompatible lubricant (e.g., petroleum jelly, oils, fats, silicone, or other lubricants).

The systems and methods disclosed herein can be modified in any suitable manner. For example, the coupler system 100 as described herein is generally presented as having an add-on coupler 110 with a coupler receptacle 111 configured to receive a portion of an existing implant. However, in some cases, the add-on coupler includes one or more processes, protrusions, or other components configured to be received into one or more receptacles of an existing implant. In other words, some embodiments of the coupling mechanisms discussed herein are reversed with respect to the configuration of the add-on coupler and the existing implant. As mentioned above, some embodiments of the add-on coupler 110 do not have a traditional receptacle configured to receive another component (such as a portion of the first coupler 120). Rather, in some such embodiments, the coupler receptacle 111 can be inverted (e.g., the coupler receptacle includes a protrusion configured to be received within a receptacle of the first coupler (for example, an anchor receptacle 121 can include an additional receptacle configured to receive the add-on coupler). By way of non-limiting illustration, FIG. 5 shows a coupling system 100 having an add-on coupler 110 with a coupler receptacle 111 in substantially the form of a threaded cylinder (protruding from the body of the add-on coupler) configured to receive (or, in this case, be received within) an anchor receptacle 121 of a first coupler 120. In other words, the add-on coupler has a coupler receptacle 111 that protrudes from, rather than recesses into, the body of the add-on coupler, thereby forming a threaded cylinder configured to screw into complementary threads formed in an anchor receptacle 121.

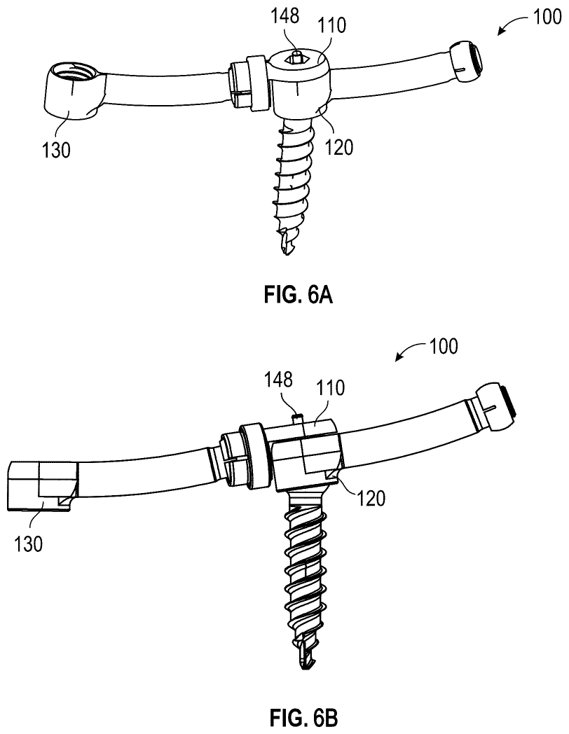

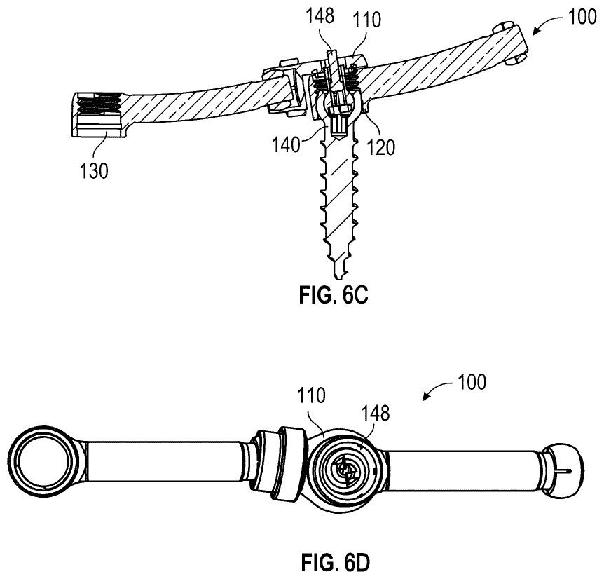

In some embodiments, the add-on coupler is substantially flat (or otherwise shaped) without either a traditional receptacle or protrusion configured to be coupled to the first coupler, but rather the add-on coupler is configured to couple to the first coupler via one or more intermediate connectors. In such cases, the intermediate connector can be any suitable connector (e.g., one or more nails, screws, bolts, staples, eyelets, magnets, hook-and-loop fasteners, adhesives, welds, interference fits, friction fits, tongue-and-groove connections, snaps, ties, rivets, stakes, wire ties, threads, or any other connectors). In some cases, the intermediate connector includes a rivet. In some embodiments, the add-on coupler 110 is configured to coupler directly to an anchor 140 (e.g., through use of an intermediate connector, such as any of the connectors listed herein). By way of non-limiting illustration, some embodiments of the add-on coupler couple directly to the anchor of the first coupler 120 using a rivet 148 with a hexalobe feature designed to resist torsion, therefore eliminating the need to couple the add-on coupler to the anchor receptacle 121 of the first coupler. By way of non-limiting illustration, FIG. 4E shows an example of a rivet 148 used in connection with some embodiments. Further, FIGS. 6A-6D show a coupler system 100 with an add-on coupler 110 having a substantially flat body configured to be coupled to a first coupler 120 via a rivet 148. In some cases, the rivet 148 couples directly to an anchor 140 (as shown in FIG. 6C).

In some embodiments using a rivet 148, the rivet is configured to provide polyaxiality. In some cases, the rivet is configured to lock the system (e.g., fixing the components in place once the desired polyaxial position is determined) by creating an interference fit. For example, in some cases, the rivet has a first position (allowing polyaxial movement) and a second position (locking the components in place), and in some cases, the second position is when the rivet is inserted farther into the anchor 140 (e.g., fully seated within the anchor). In some embodiments, polyaxiality is generated between the anchor 140 and the rod 134.

The described systems and methods can also have any suitable features, such as anything that allows for reduced damage to the patient, reduced surgery time, or otherwise improved operating procedure compared with a procedure in which an existing implant must be removed in order to install an expanded implant. In some cases, the system 100 is configured to be adjusted post operatively (e.g., many years after operation, if necessary) to add more segments, adjust component angles, or otherwise modify the surgical constructs within the patient as opposed to entirely removing the constructs and replacing them with new ones.

As another example of a feature of the described systems and methods, some embodiments, allow a surgeon to parse out the steps of a primary surgical procedure to each specific level (e.g., allowing add-on couplers 110 to be individually placed). Accordingly, such embodiments can simplify the surgeon's task and improve the outcomes when compared to having the implants be applied globally (e.g., when reducing a spondy, applying compression/distraction, selecting the insertion point for the screws, etc.).

As an additional feature, some embodiments of the described systems and methods (e.g., use of the add-on coupler 110) provide surgeons with the ability to adjust the stiffness of the rod at each specific level. In this regard, it should be noted that each spinal level might need a different stiffness so as to restore equal stability to the individual segments of the spine. After all, a healthy spine has equal stability at each spinal level. In this regard, some embodiments of the described systems and methods provide for varied stiffnesses at one or more spinal levels.

As another feature, some competing devices use one or more set screws to couple various components together. In this regard, set screws can often loosen when they are loaded-especially when they are loaded many times. That said, some embodiments of the described systems and methods allow the described add-on coupler 110 to be selectively locked to one or more components without the use of any set screws. Instead, some embodiments of the described systems and methods employ one or more geometries that must first pass through a higher energy state to then achieve a lower, unlocked state such that the add-on coupler cannot spontaneously loosen when loaded. Thus, some embodiments of the described implants are configured to provide stability and prevent spontaneous loosening.

As another example of a suitable feature, some embodiments of the described components (e.g., the add-on coupler 110) can aid in manipulating the spine by applying force through the implants to the spine. Additionally, although some competing devices are relatively large and bulky such that they get in the way of visualizing the spine during the manipulations, some embodiments of the described add-on coupler are relatively small and strong such that they can withstand the application of manipulating forces while not inhibiting visualization of critical anatomy. Indeed, some embodiments of the add-on coupler are only between 5% and 50% larger (or any subrange thereof) than the component to which the add-on coupler is added onto. For example, when some embodiments of an add-on coupler are snapped over an anchor receptacle of a first coupler, the size increase (by volume) from the anchor receptacle to the add-on coupler is between 5% and 50% (or any subrange thereof), such as 10%±5%, 25%±10%, or another suitable size increase. In some embodiments, the size increase is less than 100% (e.g., the volume of the construct with the add-on coupler is less than double the volume of the construct without the add-on coupler). In some cases, the size increase is less than 75%.

Relatedly, many existing devices require substantially more bulk to achieve the same locked strength, but the systems and methods provided herein can provide a low bulk-to-locked-strength ratio (e.g., many newtons of locked strength for comparatively few cm of bulk). For example, in some embodiments, the couplers used herein (e.g., the add-on coupler 110, the first coupler 120, the second coupler 130, or any other coupler) has a small size. For example, in some embodiments, a small-size tulip can be used, such as a tulip of less than 12 mm (or less than anywhere between 5 mm and 20 mm, or any subrange therein, such as less than 15 mm, less than 10 mm, or another suitable size). For example, in some embodiments, the system can be used with a 5.5 mm rod, yet with less than 12 mm tulip (ordinarily, the smallest predicate tulip for a 5.5 mm rod is greater than 12 mm).

As still another example of a suitable feature, some embodiments of the described add-on coupler allow for simple addition of a poly-axial extension coupler to the end of an existing construct. Indeed, while many competing devices only have one configuration, some embodiments of the described add-on coupler are poly-axial.

As an additional feature, some embodiments of the described systems and methods put one or more components under such pressure that the components flex or change their shape (flexing materials within their elastic limit)—making it very hard to change the configuration of the components once they are locked in place. Thus, some embodiments are configured to lock the poly-axial joint (e.g., between the neck 116 and the rod 124) by flexing the materials (in some cases, requiring that the materials move to a higher energy state before they can be disconnected) rather than simply relying on friction dependent mechanisms, such as a set screw.

Another example of a suitable feature is that the add-on coupler, according to some embodiments, does not sustain any permanent deformation when locked or unlocked. Accordingly, the add-on coupler can be repetitively locked and unlocked (e.g., an unlimited number of times) without decreasing the locking strength.

Another example of a suitable feature is that some embodiments include cannulated components (e.g., insertion instruments), thereby allowing for blind, percutaneous implantation. In some cases, the instruments are configured to provide tactile feedback (or other feedback) to a user when components are properly locked in place (for example, vibrations from an add-on coupler clicking into place can travel up the instrument and let a clinician know that the add-on coupler is locked in place).

As the systems and methods disclosed herein are compatible with one another, the systems discussed herein can be used in practicing the methods disclosed herein, and vice versa. Any element of any figure and any portion of any method can be combined with any other element of any figure or any other portion of any method. The systems disclosed herein can be made in any suitable manner, and they may be used in any way consistent with their operational capabilities. Moreover, in some cases, any particular element or elements of any apparatus—or portion or portions of any method—disclosed herein can be omitted. Additionally, the various components, embodiments, embodiments, cases, elements, FIGS., and other aspects described herein are not exclusive and can be combined and interchanged in any suitable manner. For instance, any aspect of one embodiment or FIG. can be combined with any other aspect of one or more embodiments or FIGS.

As used herein, the singular forms “a”, “an”, “the” and all other singular references include plural referents (e.g., one or more), and plural references include the singular (e.g., one or more), unless the context clearly requires otherwise. For example, reference to an instrument includes reference to one or more instruments, and reference to couplers includes reference to one or more couplers. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”. Additionally, the term “diameter” is not limited to a circular diameter (e.g., it may refer to a width) unless stated otherwise.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “inside”, “outside”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An add-on coupler for use in a surgical procedure, the add-on coupler comprising:
a coupler receptacle configured to receive and couple to a first coupler, wherein the first coupler is configured to couple to an anchor, wherein the anchor is configured to be implanted in a patient's body; and
a joint with at least 1 rotational degree of freedom.

2. The add-on coupler of claim 1, wherein the joint with at least 1 rotational degree of freedom comprises a rod receptacle configured to provide a poly-axial coupling to the rod.

3. The add-on coupler of claim 2, wherein the rod receptacle is connected to the coupler receptacle by a neck having a slit formed therein.

4. The add-on coupler of claim 1, wherein the add-on coupler is configured to form a press-fit connection between the add-on coupler and an adjoining component of the first coupler.

5. The add-on coupler of claim 4, further comprising a collar slidably disposed along the neck.

6. The add-on coupler of claim 5, wherein the collar is configured to at least partially compress the slit to decrease an inner diameter of the neck proximate the slit.

7. An add-on coupler for use in a surgical procedure, the add-on coupler comprising:
a body configured to couple directly to an anchor receptacle of a first coupler, the body being further configured to receive a rod of a second coupler, thereby allowing the second coupler to be mechanically joined to the first coupler via the add-on coupler.

8. The add-on coupler of claim 7, wherein the body is configured to receive the rod of the second coupler polyaxially, such that the rod can be disposed at various angles along multiple axes to provide a custom configuration for joining the second coupler to the first coupler, and wherein the body is configured to selectively lock the rod in place to prevent movement of the rod with respect to the add-on coupler.

9. A coupler system comprising:
a first anchor coupled to a first coupler at an anchor receptacle of the first coupler;
a second anchor coupled to a second coupler at an anchor receptacle of the second coupler;
an add-on coupler comprising:
a coupler receptacle configured to receive and couple to the anchor receptacle of the first coupler; and
a rod receptacle configured to receive and couple to a rod of the second coupler.

10. The coupler system of claim 9, wherein the add-on coupler further comprises an opening configured to accommodate a rod of the first coupler.

11. The coupler system of claim 10, wherein the opening increases a flexibility of the coupler receptacle.

12. The coupler system of claim 11, further comprising a process configured to decrease the flexibility of the coupler receptacle.

13. The coupler system of claim 9, wherein the rod receptacle comprises a neck having a slit formed therein.

14. The coupler system of claim 13, further comprising a collar configured to selectively compress the slit to lock the rod of the second coupler in place within the add-on coupler.

15. The coupler system of claim 9, wherein the first coupler is configured to be secured to a first spinal vertebra via a first anchor disposed in the anchor receptacle of the first coupler, and the second coupler is configured to be secured to a second spinal vertebra via a second anchor disposed in the anchor receptacle of the second coupler.

16. A method of coupling a second coupler of a spinal implant to a first coupler of the spinal implant, the method comprising:
coupling a coupler receptacle of an add-on coupler to an anchor receptacle of the first coupler; and
coupling a rod receptacle of the add-on coupler to a rod of the second coupler.

17. The method of claim 16, wherein the anchor receptacle of the first coupler is coupled to an existing anchor.

18. The method of claim 17, further comprising coupling a second anchor receptacle of the second coupler to a new anchor.

19. The method of claim 18, wherein the coupling the coupler receptacle of the add-on coupler to the anchor receptacle of the first coupler comprises inserting a rivet into the coupler receptacle and the anchor receptacle and securing the rivet in place.

20. The method of claim 16, wherein:
the coupling the coupler receptacle of the add-on coupler to the anchor receptacle of the first coupler comprises using an anchor locker; and
the coupling the rod receptacle of the add-on coupler to the rod of the second coupler comprises using a rod locker.
